(19) United States
(12) Patent Application Publication
(10) Pub. No.: US 2020/0254112 A1
(43) Pub. Date: Aug. 13, 2020

(54) ANTIBODY CONJUGATION METHOD

(71) Applicant: FUJIREBIO DIAGNOSTICS, INC., MALVERN, PA (US)

(72) Inventor: Christopher J. SPEDALIERE, Allentown, PA (US)

(73) Assignee: FUJIREBIO DIAGNOSTICS, INC., MALVERN, PA (US)

(21) Appl. No.: 16/639,393

(22) PCT Filed: Aug. 14, 2018
(86) PCT No.: PCT/US2018/046587

§ 371 Date: Feb. 14, 2020

Related U.S. Application Data
(60) Provisional application No. 62/544,977, filed on Aug. 14, 2017.

(51) Int. Cl. A61K 47/68

(52) U.S. Cl. CPC A61K 47/6811; A61K 47/6815; A61K 47/6851

(57) ABSTRACT

Provided herein are methods and materials for making antibody-polypeptide conjugates.

### FIELD OF THE INVENTION

The present invention relates to methods and compositions for making an antibody-polypeptide conjugate.

### BACKGROUND OF THE INVENTION

Antibodies have both diagnostic and therapeutic applications. Monoclonal antibodies are particularly useful because they are directed against a single epitope and can be produced in unlimited quantities. Detection of specific binding of an antibody to its target requires additional reagents and/or chemical modification of the antibody. Depending upon the antibody, the reagents and/or the modification, such detection systems can potentially impact the performance of an antibody. There is a continuing need for the development of methods of detection of specific binding of antibodies to their targets.

### SUMMARY OF THE INVENTION

Provided herein are methods of making an antibody polypeptide conjugate. The methods can include providing a first solution comprising an activated antibody; providing a second solution comprising an activated polypeptide; passing the solutions through a continuous flow reactor, wherein the activated antibody contacts the activated polypeptide, thereby forming an antibody-polypeptide conjugate. The polypeptide can be an enzyme, a biotin binding polypeptide, or a fluorescent protein. The solutions can be passed through the continuous flow reactor with a flow rate of about 0.1 mL/min to about 10.0 mL/min. The flow rate can provide a residence time of between about 30 seconds to about 15 minutes. In some embodiments the flow rate can provide a residence time of between about 1 minute to between about 5 minutes. The antibody-polypeptide conjugate can include an antibody: polypeptide ratio of from 1:1 to 1:3.

### BRIEF DESCRIPTION OF THE DRAWINGS

These and other features and advantages of the present invention will be more fully disclosed in, or rendered obvious by, the following detailed description of the preferred embodiment of the invention, which is to be considered together with the accompanying drawings wherein like numbers refer to like parts and further wherein:

FIG. 1 is a diagram illustrating one embodiment of the method.

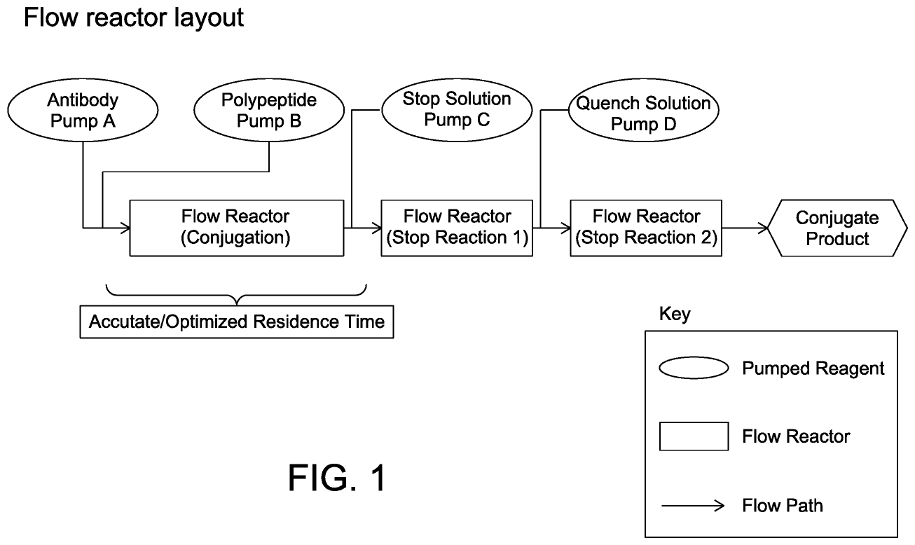

FIG. 2 is a graph depicting the results of an experiment comparing the effect of increasing residence time on covalent conjugate yield.

FIG. 3 is a graph depicting the results of an experiment comparing the effect of HRP-antibody ratio to the percentage of high molecular weight covalent conjugate.

FIG. 4 is a graph depicting the results of an experiment comparing the effect of increasing residence time on noncovalent conjugate yield.

FIG. 5 is a graph depicting the results of an experiment comparing the effect of avidin: biotinylated antibody ratio to the percentage of high molecular weight non-covalent conjugate.

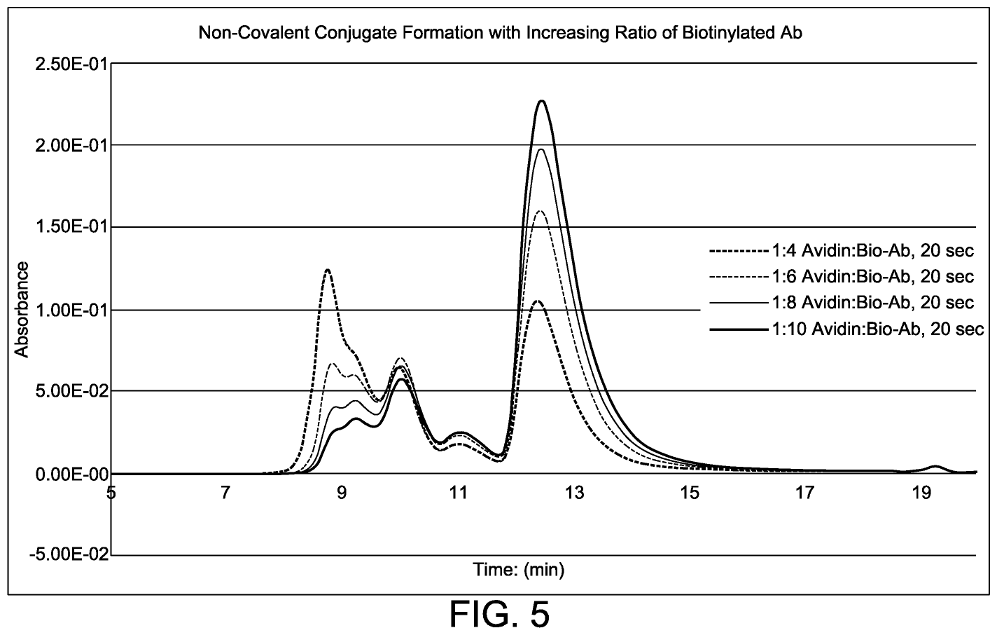

FIG. 6 is a diagram illustrating one embodiment of the method.

### DETAILED DESCRIPTION OF THE PREFERRED EMBODIMENT

This description of preferred embodiments is intended to be read in connection with the accompanying drawings, which are to be considered part of the entire written description of this invention. The drawing figures are not necessarily to scale and certain features of the invention may be shown exaggerated in scale or in somewhat schematic form in the interest of clarity and conciseness. In the description, relative terms such as “horizontal,” “vertical,” “up,” “down,” “top” and “bottom” as well as derivatives thereof (e.g., “horizontally,” “downwardly,” “upwardly,” etc.) should be construed to refer to the orientation as then described or as shown in the drawing figure under discussion. These relative terms are for convenience of description and normally are not intended to require a particular orientation. Terms including “inwardly” versus “outwardly,” “longitudinal” versus “lateral” and the like are to be interpreted relative to one another or relative to an axis of elongation, or an axis or center of rotation, as appropriate. Terms concerning attachments, coupling and the like, such as “connected” and “interconnected,” refer to a relationship wherein structures are secured or attached to one another either directly or indirectly through intervening structures, as well as both movable or rigid attachments or relationships, unless expressly described otherwise. The term “operatively connected” is such an attachment, coupling or connection that allows the pertinent structures to operate as intended by virtue of that relationship. When only a single machine is illustrated, the term “machine” shall also be taken to include any collection of machines that individually or jointly execute a set (or multiple sets) of instructions to perform any one or more of the methodologies discussed herein. In the claims, means-plus-function clauses, if used, are intended to cover the structures described, suggested, or rendered obvious by the written description or drawings for performing the recited function, including not only structural equivalents but also equivalent structures.

The present invention is based in part on the inventor's development of a method to efficiently conjugate an antibody to a polypeptide. Conjugation of antibodies to polypeptides can result in product heterogeneity, that is, inconsistency in the ratio of antibody: polypeptide in the conjugate, and the formation of high molecular weight aggregates. Both the inconsistency in the antibody: polypeptide ratio and the high molecular weight aggregates can result in variability in performance of the antibody in molecular diagnostic applications. The inventor has found that conjugation of an antibody to a detection polypeptide in a continuous flow system provided both consistent production of antibody-polypeptide conjugates having defined ratios of antibody-polypeptide and a reduction in the levels of the high molecular weight species of antibody-polypeptide conjugates and aggregates of antibody and polypeptide.

Accordingly, provided herein are materials and methods for conjugation of an antibody to a polypeptide. The methods are useful for preparing conjugated antibodies for use in immunoassays for diagnostic applications. The diagnostic applications can include diagnosis of a variety of diseases and disorders including, for example, cancer, infectious disease, autoimmune disorders, neurological disorders, and cardiovascular disorders.

### Compositions

Provided herein are materials and methods for making an antibody-polypeptide conjugate. The antibody-polypeptide conjugate can include an antibody and a polypeptide joined by a covalent bond. In some embodiments, the conjugate can include two or more polypeptides joined to the antibody. In some embodiments, the antibody-polypeptide conjugate can include an antibody and a polypeptide joined by a noncovalent bond, for example, an ionic bond.

### Antibodies

We use the term antibody to broadly refer to immunoglobulin-based binding molecules, and the term encompasses conventional antibodies (e.g., the tetrameric antibodies of the G class (e.g., an IgG₁)), fragments thereof that retain the ability to bind their intended target (e.g., an Fab′ fragment), and single chain antibodies (scFvs). The antibody may be polyclonal or monoclonal and may be produced by human, mouse, rabbit, sheep or goat cells, or by hybridomas derived from these cells. In some embodiments, the antibody can be humanized, or chimeric.

The antibodies can assume various configurations and encompass proteins consisting of one or more polypeptides substantially encoded by immunoglobulin genes. Any one of a variety of antibody structures can be used, including the intact antibody, antibody multimers, or antibody fragments or other variants thereof that include functional, antigen-binding regions of the antibody. We may use the term “immunoglobulin” synonymously with “antibody.” The antibodies may be monoclonal or polyclonal in origin. Regardless of the source of the antibody, suitable antibodies include intact antibodies, for example, IgG tetramers having two heavy (H) chains and two light (L) chains, single chain antibodies, chimeric antibodies, humanized antibodies, complementary determining region (CDR)-grafted antibodies as well as antibody fragments, e.g., Fab, Fab′, F(ab′)₂, scFv, Fv, and recombinant antibodies derived from such fragments, e.g., camelbodies, microantibodies, diabodies and bispecific antibodies.

An intact antibody is one that comprises an antigen-binding variable region (V_{H} and V_{L}) as well as a light chain constant domain (C_{L}) and heavy chain constant domains, C_{H1}, C_{H2} and C_{H3}. The constant domains may be native sequence constant domains (e.g. human native sequence constant domains) or amino acid sequence variants thereof. The V_{H} and V_{L} regions are further subdivided into regions of hypervariability, termed “complementarity determining regions” (CDRs), interspersed with the more conserved framework regions (FRs). The CDR of an antibody typically includes amino acid sequences that together define the binding affinity and specificity of the natural Fv region of a native immunoglobulin binding site.

The antibody can be from any class of immunoglobulin, for example, IgA, IgG, IgE, IgD, IgM (as well as subtypes thereof (e.g., IgG₁, IgG₂, IgG₃, and IgG₄)), and the light chains of the immunoglobulin may be of types kappa or lambda. Human immunoglobulin genes include the kappa, lambda, alpha (IgA₁ and IgA₂), gamma (IgG₁, IgG₂, IgG₃, IgG₄), delta, epsilon, and mu constant region genes, as well as the many immunoglobulin variable region genes.

The term “antigen-binding portion” of an immunoglobulin or antibody refers generally to a portion of an immunoglobulin that specifically binds to a target. An antigen-binding portion of an immunoglobulin is therefore a molecule in which one or more immunoglobulin chains are not full length, but which specifically binds to a target. Examples of antigen-binding portions or fragments include: (i) an Fab fragment, a monovalent fragment consisting of the VLC, VHC, CL and CH1 domains; (ii) a F(ab′)₂ fragment, a bivalent fragment comprising two Fab fragments linked by a disulfide bridge at the hinge region; (iii) a Fv fragment consisting of the VLC and VHC domains of a single arm of an antibody, and (v) an isolated CDR having sufficient framework to specifically bind, e.g., an antigen binding portion of a variable region. An antigen-binding portion of a light chain variable region and an antigen binding portion of a heavy chain variable region, e.g., the two domains of the Fv fragment, VLC and VHC, can be joined, using recombinant methods, by a synthetic linker that enables them to be made as a single protein chain in which the VLC and VHC regions pair to form monovalent molecules (known as single chain Fv (scFv). Such scFvs are encompassed by the term “antigen-binding portion” of an antibody.

An “Fv” fragment is the minimum antibody fragment that contains a complete antigen-recognition and binding site. This region consists of a dimer of one heavy chain and one light chain variable domain in tight, con-covalent association. It is in this configuration that three hypervariable regions of each variable domain interact to define an antigen-binding site on the surface of the VH-VL dimer. While six hypervariable regions confer antigen-binding specificity, even a single variable domain (or half of an Fv comprising only three hypervariable regions specific for an antigen) has the ability to recognize and bind antigen, although at a lower affinity than the entire binding site. To improve stability, the VH-VL domains may be connected by a flexible peptide linker such as (Gly₄Ser)₃ to form a single chain Fv or scFV antibody fragment or may be engineered to form a disulfide bond by introducing two cysteine residues in the framework regions to yield a disulfide stabilized Fv (dsFv).

Fragments of antibodies are suitable for use in the methods provided so long as they retain the desired specificity of the full-length antibody. Methods for preparing antibody fragments encompass both biochemical methods (e.g. proteolytic digestion of intact antibodies which may be followed by chemical cross-linking) and recombinant DNA-based methods in which immunoglobulin sequences are genetically engineered to direct the synthesis of the desired fragments. Antibody fragments can be obtained by proteolysis of the whole immunoglobulin by the non-specific thiolprotease, papain. Papain digestion yields two identical antigen-binding fragments, termed “Fab fragments,” each with a single antigen-binding site, and a residual “Fc fragment.” The various fractions can be separated by protein A-Sepharose or ion exchange chromatography. A typical procedure for preparation of F(ab′)₂ fragments from IgG of rabbit and human origin is limited proteolysis by the enzyme pepsin. Pepsin treatment of intact antibodies yields an F(ab′)₂ fragment that has two antigen-combining sites and is still capable of cross-linking antigen. A Fab fragment contains the constant domain of the light chain and the first constant domain (CH1) of the heavy chain. Fab′ fragments differ from Fab fragments by the addition of a few residues at the carboxyl terminus of the heavy chain CH1 domain including one or more cysteine(s) from the antibody hinge region. F(ab′)₂ antibody fragments were originally produced as pairs of Fab′ fragments that have hinge cysteines between them.

Monoclonal antibodies are homogeneous antibodies of identical antigenic specificity produced by a single clone of antibody-producing cells. Polyclonal antibodies generally recognize different epitopes on the same antigen and are produced by more than one clone of antibody producing cells. Each monoclonal antibody is directed against a single determinant on the antigen. The modifier, monoclonal, indicates the character of the antibody as being obtained from a substantially homogeneous population of antibodies, and is not to be construed as requiring production of the antibody by any particular method.

Monoclonal antibodies can be chimeric antibodies, i.e., antibodies that typically have a portion of the heavy and/or light chain identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity. Chimeric antibodies can all include primatized antibodies comprising variable domain antigen-binding sequences derived from a non-human primate (e.g. apes, Old World monkeys, New World monkeys, prosimians) and human constant region sequences.

Humanized antibodies are generally chimeric or mutant monoclonal antibodies from mouse, rat, hamster, rabbit or other species, bearing human constant and/or variable region domains or specific changes. The framework of the immunoglobulin can be human, humanized, or non-human (e.g., a murine framework modified to decrease antigenicity in humans), or a synthetic framework (e.g., a consensus sequence). Humanized immunoglobulins are those in which the framework residues correspond to human germline sequences and the CDRs result from V(D)J recombination and somatic mutations. However, humanized immunoglobulins may also comprise amino acid residues not encoded in human germline immunoglobulin nucleic acid sequences (e.g., mutations introduced by random or site-specific mutagenesis ex vivo). An antibody variable domain gene based on germline sequence but possessing framework mutations introduced by, for example, an in vivo somatic mutational process is termed “human.”

The antibody may be modified. In some embodiments, the antibody can be modified to reduce or abolish glycosylation. An immunoglobulin that lacks glycosylation may be an immunoglobulin that is not glycosylated at all; that is not fully glycosylated; or that is atypically glycosylated (i.e., the glycosylation pattern for the mutant differs from the glycosylation pattern of the corresponding wild type immunoglobulin). The IgG polypeptides include one or more (e.g., 1, 2, or 3 or more) mutations that attenuate glycosylation, i.e., mutations that result in an IgG CH2 domain that lacks glycosylation, or is not fully glycosylated or is atypicially glycosylated. The oligosaccharide structure can also be modified, for example, by eliminating the fucose moiety from the N-linked glycan. In some embodiments, the antibody can also be modified to increase the stability and or solubility by conjugation to non-protein polymers, e.g, polyethylene glycol. In some embodiments, the antibody can be modified via biotinylation, carboxylation, phosphorylation, methylation, acetylation, nitrosylation, citrullination or deamination.

Useful antibodies specifically bind to an epitope present on a target. The specific target can vary. Exemplary classes of targets include polypeptides, carbohydrates, lipids, nucleic acids, or small molecules such as metabolites or therapeutics. An epitope refers to an antigenic determinant on a target that is specifically bound by the paratope, i.e., the binding site of an antibody. Epitopic determinants usually consist of chemically active surface groupings of molecules such as amino acids or sugar side chains, and typically have specific three-dimensional structural characteristics, as well as specific charge characteristics. In the case of polypeptide targets, epitopes generally have between about 4 to about 10 contiguous amino acids (a linear or continuous epitope), or alternatively can be a set of noncontiguous amino acids that define a particular structure (e.g., a conformational epitope). Thus, an epitope can consist of at least 4, at least 6, at least 8, at least 10, and at least 12 such amino acids. Methods of determining the spatial conformation of amino acids can include, for example, x-ray crystallography and 2-dimensional nuclear magnetic resonance.

A useful antibody exhibits a threshold level of binding activity; and/or does not significantly cross-react with known related polypeptide molecules. In some embodiments, the antibody can bind to the target epitopes or mimetic decoys at least 1.5-fold, 2-fold, 5-fold 10-fold, 100-fold, 10³-fold, 10⁴-fold, 10⁵-fold, 10⁶-fold or greater for the target than to other proteins predicted to have some homology to the target.

In some embodiments the antibody binds with high affinity of 10⁻⁴ M or less, 10⁻⁷ M or less, 10⁻⁹M or less or with subnanomolar affinity (0.9, 0.8, 0.7, 0.6, 0.5, 0.4, 0.3, 0.2, 0.1 nM or even less). In some embodiments the binding affinity of the antibody for its respective target is at least 1×10⁶ Ka. In some embodiments the binding affinity of the antibody for its target is at least 5×10⁶ Ka, at least 1×10⁷ Ka, at least 2×10⁷ Ka, at least 1×10⁸ Ka, or greater. An antibody may also be described or specified in terms of the binding affinity to its target. In some embodiments, the antibody has a binding affinity with a Kd less than 5×10⁻² M, 10⁻² M, 5×10⁻³ M, 10⁻³ M, 5×10⁻³M, 10⁻⁴ M, 5×10⁻⁵ M, 10⁻⁵ M, 5×10.⁻⁶ M, 10⁻⁶ M, 5×10⁻⁷ M, 10⁻⁷ M, 5×10⁻⁸ M, 10⁻⁸M, 5×10⁻⁹ M, 5×10⁻¹⁰ M, 10⁻¹⁰ M, 5×10⁻¹¹ M, 10⁻¹¹M, 5×10⁻¹²M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M, or less.

As noted, the antibody can be an antibody that specifically binds to a target. The target can be, for example, a biomarker, also referred to as a marker, related to a disease or disorder. Exemplary diseases or disorders include cancer, cardiovascular disease, inflammation, neurological disorders such as Alzheimer's disease and other dementias, urinary disorders, bone disorders, pulmonary disorders, gastrointestinal disorders, reproductive disorders, muscle disorders, lymphatic disorders, immune system disorders, and infectious diseases. The biomarker can be a predictive, prognostic, diagnostic or surrogate biomarker for a disease or a disorder. The biomarker can be present in a biological sample, for example, a body fluid, blood, serum, plasma, urine, semen, cerebrospinal fluid, saliva, tear, mucus, synovial fluid, breast milk, interstitial fluid, feces, lymph, bile or vaginal secretion. In some embodiments the biological sample can be a tissue sample.

The antibody can be an antibody that specifically binds to a target expressed by a cancer cell. Exemplary cancers include, without limitation, hematological cancers such as leukemias and lymphomas, neurological tumors such as astrocytomas or glioblastomas, melanoma, breast cancer, lung cancer, head and neck cancer, thyroid cancer, gastrointestinal tumors such as gastric or colon cancer, liver cancer, pancreatic cancer, genitourinary tumors such ovarian cancer, vaginal cancer, uterine cancer, bladder cancer, testicular cancer, prostate cancer or penile cancer, bone tumors, and vascular tumors.

In some embodiments, the target can be a tumor-associated antigen (TAA). A TAA can be a molecule (e.g., a polypeptide, carbohydrate or lipid) that is expressed by a tumor cell and either (a) differs qualitatively from its counterpart expressed in normal cells, or (b) is expressed at a higher level in tumor cells than in normal cells. Thus, a TAA can differ (e.g., by one or more amino acid residues where the molecule is a protein) from, or it can be identical to, its counterpart expressed in normal cells. Preferably it is not expressed by normal cells. Alternatively, it is expressed at a level at least two-fold higher (e.g., a two-fold, three-fold, five-fold, ten-fold, 20-fold, 40-fold, 100-fold, 500-fold, 1000-fold, 5000-fold, or 15000-fold higher) in a tumor cell than in the tumor cell's normal counterpart. Exemplary cancer cell targets include CA-125, HE4, carcinoembryonic antigen (CEA), MUC (mucin, for example, MUC1), prostate-specific antigen (PSA), carbohydrate antigen 15.3 (CA 15-3), estrogen receptor (ER), progesterone receptor (PgR), HER2, carbohydrate antigen 27.29 (CA 27.29), human chorionic gonadotropin-β (HCG-β), a-fetoprotein, calcitonin, thyroglobulin, CA 19-9, nuclear matrix protein 22 (NMP-22), prostate cancer antigen 3 (PSA₃), Epstein-Barr Virus nuclear antigen, and human papilloma virus (HPV) E6 and E7.

In some embodiments, the antibody can be an antibody that specifically binds to a molecule expressed or released by any of a wide range of infectious agents, including, without limitation, viruses, viroids, bacteria, fungi, prions or parasites. For example, viral pathogens can include, without limitation, influenza viruses, including the strain A (H1N5), hepatitis viruses (e.g, Hepatitis A, B, C and D), Arenaviruses, Bunyaviruses, Flaviviruses, Filoviruses, Alphaviruses, (e.g., Venezuelan equine encephalitis, eastern equine encephalitis, western equine encephalitis), Hantaviruses, human immunodeficiency viruses HIV1 and HIV2, feline immunodeficiency virus, simian immunodeficiency virus, measles virus, rabies virus, rotaviruses, papilloma virus, respiratory syncytial virus, Variola, and viral encephalitides, (e.g., West Nile Virus, LaCrosse, California encephalitis, VEE, EEE, WEE, Japanese Encephalitis Virus, Kyasanur Forest Virus). Bacterial pathogens can include, but are not limited to, Bacillus anthracis, Yersinia pestis, Yersinia enterocolitica, Clostridium botulinum, Clostridium perfringens Francisella tularensis, Brucella species, Salmonella spp., including Salmonella enteriditis, Escherichia coli including E. coli O157:H7, Streptococcus pneumoniae, Staphylococcus aureus, Burkholderia mallei, Burkholderia pseudomallei, Chlamydia spp., Coxiella burnetii, Rickettsia prowazekii, Vibrio spp., Shigella spp. Listeria monocytogenes, Mycobacteria tuberculosis, M. leprae, Borrelia burgdorferi, Actinobacillus pleuropneumoniae, Helicobacter pylori, Neisseria meningitidis, Bordetella pertussis, Porphyromonas gingivalis, and Campylobacter jejuni.

Fungal pathogens can include, without limitation, members of the genera Aspergillus, Penecillium, Stachybotrys, Trichoderma, mycoplasma, Histoplasma capsulatum, Cryptococcus neoformans, Chlamydia trachomatis, and Candida albicans.

Pathogenic protozoa can include, for example, members of the genera Cryptosporidium, e.g., Cryptosporidium parvum, Giardia lamblia, Microsporidia and Toxoplasma, e.g., Toxoplasma brucei, Toxoplasma gondii, Entamoeba histolytica, Plasmodium falciparum, Leishmania major and Cyclospora cayatanensis.

### Polypeptides

The compositions of the invention can include a polypeptide, for example, a polypeptide that permits the detection of specific binding of the antibody to its target. The terms “peptide,” “polypeptide,” and “protein” are used interchangeably herein, although typically they refer to peptide sequences of varying sizes. We may refer to the amino acid-based compositions of the invention as “polypeptides” to convey that they are linear polymers of amino acid residues, and to help distinguish them from full-length proteins. A polypeptide of the invention can “constitute” or “include” a fragment of a polypeptide, provided it retained sufficient activity to permit detection of specific binding of the antibody to its target. Polypeptides can be generated by a variety of methods including, for example, recombinant techniques or chemical synthesis.

The polypeptide can be a detection polypeptide, that is, a polypeptide, which when conjugated to the antibody, facilitates detection of specific binding of the antibody to its target. Detection polypeptides can include enzymes, biotin binding polypeptides, and fluorescent proteins. Useful enzymes include horseradish peroxidase, alkaline phosphatase, urease, soy bean peroxidase, beta-lactamase, beta galactosidase, and glucose oxidase. The biotin-binding polypeptide can be any polypeptide that specifically binds non-covalently to biotin or a biotin mimetic, for example, streptavidin, avidin, neutravidin or an anti-biotin antibody. In some embodiments, the polypeptide can be modified via glycosylation, carboxylation, phosphorylation, methylation, acetylation, deacetylation, nitrosylation, citrullination and deimination.

The antibody and the polypeptide, for example, the detection polypeptide can be purified prior to conjugation. The antibody and the polypeptide can be purified, for example, using filtration, centrifugation and various chromatographic methods, such as reversed phase or normal phase HPLC, size exclusion, affinity chromatography, gel filtration, hydrophobic chromatography, tangential ultrafiltration, diafiltration, ion exchange chromatography, partition chromatography on polysaccharide gel media such as Sephadex G- or affinity chromatography. These purification techniques each involve fractionation to separate the desired antibody or polypeptide from other components of a mixture. Antibodies can also be purified, for example, by protein A-Sepharose and/or protein G-Sepharose chromatography. The purity of the antibody and the polypeptide can be analyzed a variety of methods including spectrophotometric methods. The term “essentially pure” refers to chemical purity of an antibody or a polypeptide that may be substantially or essentially free of other components which normally accompany or interact with the antibody or polypeptide prior to purification. By way of example only, the antibody or polypeptide may be “essentially pure” when the preparation of the antibody or polypeptide contains less than about 30%, less than about 25%, less than about 20%, less than about 15%, less than about 10%, less than about 5%, less than about 4%, less than about 3%, less than about 2%, or less than about 1% (by dry weight) of contaminating components. For example, purification may reduce the amount of one or more of the unconjugated reactants or aggregates to 10% or less, 5% or less, or 1% or less of the amount of unconjugated reactant or aggregate that was originally present. Thus, an “essentially pure” antibody or polypeptide may have a purity level of about 70%, about 75%, about 80%, about 85%, about 90%, about 95%, about 96%, about 97%, about 98%, about 99% or greater.

### Methods

The antibody and the polypeptide are conjugated in a continuous flow device, for example, a continuous flow reactor. A variety of conjugation chemistries can be used. In general, in order to form a covalent bond between the antibody and the polypeptide, either the antibody or the polypeptide or both are activated. An activating reagent can be any reagent suitable to initiate coupling of the antibody and the polypeptide. In general, biochemical conjugation can take place through a covalent linkages at four targets within a polypeptide: 1) primary amines (—NH2) found at the N-terminus of polypeptides as well as in the side chain of lysine residues; 2) carboxyls (—COON) found at the C-terminus and in the side chains of aspartic acid and glutamic acid residues; 3) sulfhydryls (—SH) found that the side chain of cysteine residues; and 4) carbonyls (—CHO) created by oxidizing carbohydrate groups in the glycoproteins. A variety of reagents can be used to cross-link polypeptides at these residues.

In some embodiments, the antibody can be covalently linked to the polypeptide through the side chain of lysine, which terminates in a primary amine (—NH2). Amine-reactive reagents include reactive esters, such N-hydroxy succinimide (NHS) esters, iosthiocyanates, aldehydes, anhydrides, for example, diethyltriaminepentaacetic anhydride (DTPA). In some embodiments, the antibody can be covalently linked to the polypeptide through thiol active reagents, such as haloacetyl derivatives and maleimides. In some embodiments, the antibody can be covalently linked to the polypeptide through aldehyde and carboxylic acids, using carbodiimides, for example, 1-ethyl-3-(3-dimethylaminopropyl)carbodiimide hydrochloride (EDC). In some embodiments the antibody, can be covalently linked to the polypeptide through sodium periodate. In some embodiments, the antibody can be covalently linked to the polypeptide via a heterobifunctional reagent, for example, succinimidyl 6-(N-maleimido) hexanoate.

In some embodiments, the primary amine groups on the polypeptide can be converted into protected sulfhydro groups to facilitate heterobifunctional cross-linking. For example, SATA (N-succinimidyl S-acetylthioacetate) adds sulfhydryl groups to proteins and other amine-containing molecules in a protected form. SATA contains an N-hydroxysuccinimide (NHS) ester, which forms a stable, covalent amide bond with primary amines (i.e., lysine residues and the amino termini of proteins) and releases NHS as a by-product. De-protection (deacylation) to generate a free sulfhydryl can be carried out by treatment with hydroxylamine-HCI.

In some embodiments, the antibody and the polypeptide can be conjugated using a “hinge method.” The antibody is digested with pepsin to produce an F(ab′)₂ fragment. The F(ab′)₂ fragment is reduced to produce an F(ab′) fragment having a free thiol group. The polypeptide, for example, alkaline phosphatase is combined with a heterobifunctional linker containing a thiol selective maleimide group at one end and an amino group selective N-hydroxy succinimide ester at the other end. These two functional groups can be separated by a spacer. The amino group selective N-hydroxy succinimide ester reacts with the free amino group in the alkaline phosphatase to form alkaline phosphatase-maleimide. The alkaline phosphatase-maleimide is then combined with the F(ab′) fragment under the appropriate reaction conditions to produce an F(ab′) fragment covalently linked to alkaline phosphatase.

In some embodiments, the antibody and the polypeptide can be conjugated using a “non-hinge method.” The antibody is reacted with an iminothiolane to form an IgG-SH, that is an antibody having a thiol group linked to one or more primary amines. As described above, the polypeptide, for example, alkaline phosphatase is combined with a heterobifunctional linker containing a thiol selective maleimide group at one end and an amino group selective N-hydroxy succinimide ester at the other end. These two functional groups can be separated by a spacer. The amino group selective N-hydroxy succinimide ester reacts with the free amino group in the alkaline phosphatase to form alkaline phosphatase-maleimide. The alkaline phosphatase-maleimide is then combined with the IgG-SH under the appropriate reaction conditions to produce an IgG-SH covalently linked to alkaline phosphatase.

In some embodiments, the conjugate can be configured so that the antibody and the polypeptide are separated by a structural spacer. A structural spacer can be of varying length and chemical composition depending upon the cross-linker used. In some embodiments, the spacer segment can be one or more amino acids.

In some embodiments, the antibody-polypeptide conjugate can include an antibody and a polypeptide joined by a noncovalent bond, for example, an ionic bond. Thus, the antibody can be modified and then contacted with a polypeptide that specifically binds the modified antibody. Exemplary modifications include biotinylation, carboxylation, phosphorylation, methylation, acetylation, nitrosylation, citrullination or deamination. For example, the antibody can be biotinylated. The biotinylated antibody solution can be passed through a continuous flow reactor along with a solution containing a biotin binding polypeptide to form an antibody-biotin-avidin conjugate.

Generally, a continuous flow device is an integrated system of one or more chambers, ports, and channels that are interconnected and in fluid communication and designed for carrying out an analytical reaction or process. Continuous flow systems can also include instrumentation that provides support functions, such as sample introduction, fluid and/or reagent driving means, temperature control, detection systems, data collection and/or integration systems. Continuous flow devices can further include valves, pumps, and specialized functional coatings on interior walls, e.g., to prevent adsorption of sample components or reactants, and facilitate reagent movement by electroosmosis. Continuous flow devices are typically fabricated in or as a solid substrate, which may be glass, plastic, or other solid polymeric materials, and typically have a planar format for ease of detecting and monitoring sample and reagent movement, especially via optical or electrochemical methods. Continuous flow devices can have a broad range of cross-sectional dimensions. The dimensions of the device can be scaled to reaction volumes and residence times. Exemplary residence times can range from 30 seconds to 10 minutes. Flow rates are generally dependent on the dimensions of the particular system. Useful continuous flow systems can have a flow rate that provides for rapid mixing of the reagents to initiate (start) and terminate (stop) the reaction, and a well-controlled residence time, that is the time between initiation of the reaction and termination of the reaction. In general, reagents in a continuous flow device can mix by diffusion, turbulent mixing, or via an in-line static mixer.

Exemplary volumes for rapid mixing are in the microliter to milliliter range. The internal diameter of the flow path is typically in the millimeter range. A continuous flow device can have cross-sectional dimensions of less than a few hundred square micrometers and passages typically have capillary dimensions, e.g., having maximal cross-sectional dimensions of from about 500 μm to about 0.1 μm. Microfluidics devices typically have volume capacities in the range of from 1 μL to a fewer than 10 nL, e.g., 10-100 nL. Exemplary volume capacities can include 10 nL, 20 nL, 30 nL, 40 nL, 50 nL 60 nL, 70 nL, 80 nL, 90 nL, 100 nL, 120 nL, 130 nL, 140 nL, 150 nL, 160 nL, 170 nL, 180 nL, 190 nL, 200 nL, 220 nL, 240 nL, 250 nL, 300 nL, 3500 nL, 400 nL, 450 nL, 500 nL, 600 nL, 700 nL, 800 nL, 900 nL, 1000 nL,

Useful commercially available continuous flow systems for the methods disclosed herein include, the Corning Low-FIow™ Reactor, the VapourTec R-Series or E-Series systems; the Lonza FlowPlate™ series of systems; the Syrris Asia, Titan, Africa, or Dolomite Flow Systems; the Chemtrix Labtrix®, GRAMFLOW®, KILOFPOW®, PROTRIX®, PLANTRIX® OR 3D PRINTED FLOW systems. Alternatively, the continuous flow system can be a custom-built or designed continuous flow device. The dimensions of each system are scaled for a specific range of flowrates and residence times. Exemplary residence times can range from 30 seconds to 10 minutes. Flow rates are generally dependent on the dimensions of the particular system. The internal diameter of the flow path is typically in the millimeter range. Choosing and customizing these systems allows for great variability in the volume, reactant ratios, and flow-rates necessary to perform antibody-polypeptide conjugation reactions. Regardless of the specific device, useful systems will provide a flow rate that permits rapid mixing of the reagents and concomitant rapid initiation and termination of the reaction. Useful configurations allow for substantially instantaneous mixing of the reagents, exemplary volumes for rapid mixing are in the microliter to milliliter range. The rapid initiation and termination of the reaction results in more uniform conjugates and reduces the production of high molecular weight aggregates.

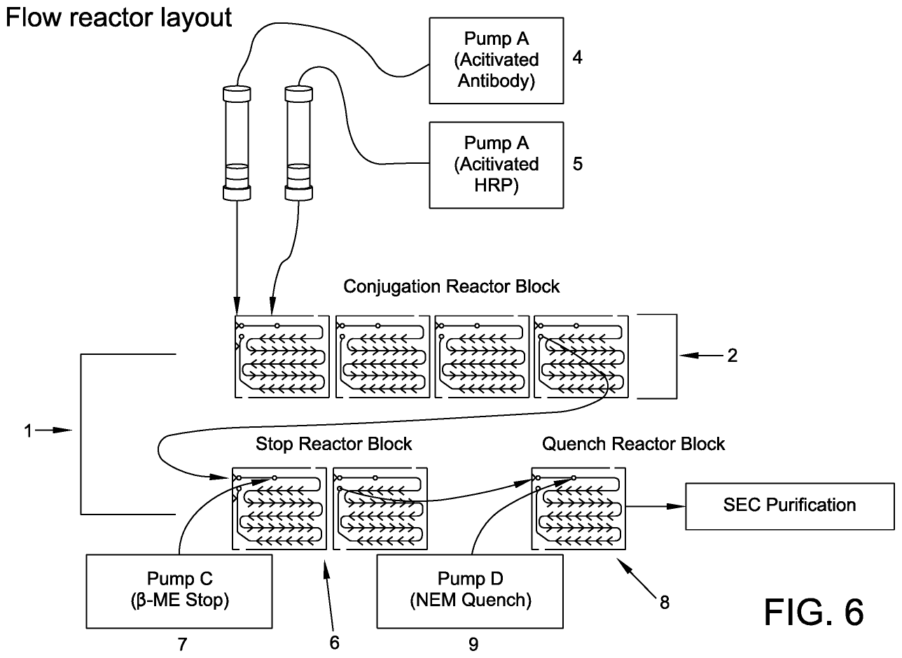

An exemplary continuous flow reactor configuration is shown in FIG. 6. The flow reactor 1 includes a conjugation reactor block 2 comprising a plurality of microchannels 3. The activated antibody is pumped into the reactor at Pump A 4. The activated HRP is pumped into the reactor at Pump B 5. The activated antibody and the activated HRP flow through the conjugation reactor block 2, where conjugation takes place. The solution then flows through the stop reactor block 6, where the 13-mercaptoethanol stop solution is pumped into the stop reactor block 6 at Pump C 7. The solution then flows through the quench reactor block 8, where the NEM quench solution is pumped into the quench reactor block 8 at Pump D 9. The solution then exits the quench reactor block 8 where it is collected.

The amount of antibody and polypeptide can vary depending upon the specific conjugation chemistry and the scale of the microreactor. The molar ratio of antibody to polypeptide can also vary. The range of molar ratios of antibody to polypeptide can vary from about 1:20 to about 20:1. Exemplary ratios include 1:20; 1:10; 1:5; 1:4; 1:2; 1:1; 2:1; 4:1, 5:1; 6:1; 10:1; 14:1; 17:1; and 20:1.

The rate at which the activated antibody and the activated polypeptide pass through the flow reactor can also vary and is dependent in part on the reaction scaled desired and the particular system used. The reaction can be scaled from quantities in the milligram range to quantities in the kilogram range. Generally, the flow rate can be between about 0.1 mL/min to about 10.0 mL/min. In some embodiments, the flow rate can be between about 1.0 mL/min to about 5.0 mL/min, between about 2.0 mL/min to about 4.0 mL/min. Thus the flow rate can be about 0.1 mL/min, about 0.5 mL/min, about 1.0 mL/min, about 1.2 mL/min, about 1.5 mL/min, about 1.8 mL/min, about 2.0 mL/min, about 2.2 mL/min, about 2.5 mL/min, about 2.8 mL/min, about 3.0 mL/min, about 3.2 mL/min, about 3.5 mL/min, about 3.8 mL/min, about 4.0 mL/min, about 4.2 mL/min, about 4.5 mL/min, about 4.8 mL/min, about 5.0 mL/min, about 5.2 mL/min, about 5.5 mL/min, about 5.8 mL/min, about 6.0 mL/min, about 6.2 mL/min, about 6.5 2 mL/min, about 6.8 mL/min, about 7.0 mL/min, about 7.2 mL/min, about 7.5 mL/min, about 7.8 mL/min, about 8.0 mL/min, about 8.2 mL/min, about 8.5 mL/min, about 8.8 mL/min, about 9.0 mL/min, about 9.2 mL/min, about 9.5 mL/min, about 9.8 mL/min, about 10.0 mL/min, about 10.5 mL/min, about 11.0 mL/min, about 11.5 mL/min, about 12.0 mL/min, about 12.5 mL/min, about 13.0 mL/min, about 13.5 mL/min, about 14.0 mL/min, about 14.5 mL/min, or about 15.0 mL/min.

Accordingly, residence time (also referred to as retention time) can also vary. The residence time of the reagents in the reactor is generally calculated based on the volume of the reactor and the flow rate such that Residence time=Reactor Volume/Flow Rate. Longer residence times can be achieved by pumping reagents more slowly and or using a larger reactor volume. Residence times can include, for example, about 20 seconds, about 30 seconds, about 40 seconds, about 50 seconds, about 60 seconds, about 70 seconds, about 80 seconds, about 90 seconds, about 100 seconds, about 120 seconds, about 140 seconds, about 150 seconds, about 160 seconds, about 170 seconds, about 180 seconds, about 190 seconds, about 200 seconds or about 1 minute, about 2 minutes, about 3 minutes, about 4 minutes, about 5 minutes, about 6 minutes, about 7 minutes, about 8 minutes, about 9 minutes, about 10 minutes, about 12 minutes, or about 15 minutes.

The reaction temperature can also vary depending upon the specific conjugation chemistry. Reaction temperatures can range from about 4° C. to about 45° C.; from about 10° C. to about 40° C., from about 15° C. to about 37° C.; from about 15° C. to about 25° C.; from about 20° C. to about 22° C.

In general, the conjugation reaction can be carried out in an aqueous solution. Buffering systems that maintain the pH in the physiological range can be used. Useful buffering systems do not react with the active groups involved in the conjugation reaction. In some embodiments, the pH can range from slightly acidic to slightly basic. For example, the pH can be about 6.0, about 6.2, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, about 8.0, about 8.1, about 8.2, about 8.3, about 8.5, about 8.8, or about 8.9.

The conjugation reaction can be terminated in a variety of ways depending upon the specific reactants. For covalent conjugations, the reaction can be terminated for example, by the addition of a reducing agent such as β mercaptoethanol. The covalent reaction can be quenched using N-ethylmaleimide. For noncovalent conjugations, the reaction can be terminated with the addition of biotin.

The concentration of conjugate in the crude conjugate solution can be determined by a variety of methods. Exemplary methods including spectrophotometric determination by measuring the absorbance at 280 nm. When the polypeptide is horseradish peroxidase, the conjugate can also be analyzed spectrophotometrically by measuring the absorbance at both 280 nm and 403 nm, which detects the prosthetic heme group on the peroxidase. The R_{z} value for the conjugate, that is, the ratio of absorbance at 403 nm to absorbance at 280 nm can be calculated. Useful R_{z} values for conjugated horseradish peroxidase range from about 0.8 to about 2.0. This corresponds to an average value of 3:1 to 4:1, HRP:Antibody. This ratio can also be determined using other methods, for example, Multi-Angle Light Scattering (MALS), Liquid Chromatography, or mass spectrometry.

The antibody-polypeptide conjugate can be purified using the methods described above, for example, filtration and size exclusion chromatography.

The antibody-polypeptide conjugates can be used in a variety of immunoassay formats. The immunoassays can include both homogeneous and heterogeneous assays, competitive and non-competitive assays, direct and indirect assays, and “sandwich” assays. Useful formats include, but are not limited to, enzyme immunoassays, for example, enzyme linked immunosorbent assays (ELISA), chemilum inescent immune-assays (CLIA), electrochem ilum inescent assays, radioimmunoassay, immunofluorescence, fluorescence anisotropy, immunoprecipitation, equilibrium dialysis, immunodiffusion, immunoblotting, agglutination, luminescent proximity assays, and nephelometry.

Regardless of the format, the biological sample is contacted with an antibody. In some embodiments, the biological sample can be immobilized on a solid support. In some embodiments, the biological sample is contacted with an antibody that has been immobilized on a solid support. The solid support can be, for example, a plastic surface, a glass surface, a paper or fibrous surface, or the surface of a particle. More specifically, the support can include a microplate, a bead, a polyvinylidene difluoride (PVDF) membrane, nitrocellulose membrane, nylon membrane, porous membranes, non-porous membranes. The composition of the substrate can be varied. For example, substrates or support can comprise glass, cellulose-based materials, thermoplastic polymers, such as polyethylene, polypropylene, or polyester, sintered structures composed of particulate materials (e.g., glass or various thermoplastic polymers), or cast membrane film composed of nitrocellulose, nylon, or polysulfone. In general embodiments, the substrate may be any surface or support upon which an antibody or a polypeptide can be immobilized, including one or more of a solid support (e.g., glass such as a glass slide or a coated plate, silica, plastic or derivatized plastic, paramagnetic or non-magnetic metal), a semi-solid support (e.g., a polymeric material, a gel, agarose, or other matrix), and/or a porous support (e.g., a filter, a nylon or nitrocellulose membrane or other membrane). In some embodiments, synthetic polymers can be used as a substrate, including, e.g., polystyrene, polypropylene, polyglycidylmethacrylate, aminated or carboxylated polystyrenes, polyacrylam ides, polyamides, and polyvinylchlorides.

Antibody binding can be measured in a variety of ways. The signal, for example, the signal generated by a detectable label, can be analyzed and, if applicable, quantified using an optical scanner or other image acquisition device and software that permits the measurement of the signal, for example a fluorescent signal a luminescent signal, or a phosphorescent signal, or a radioactive signal, associated with complex formation. Exemplary instrumentation for measuring a detectable signal can include, but is not limited to microplate readers, fluorimeters, spectrophotometers, and gamma counters.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1

### Materials and Methods

Antibody preparation and activation. Anti-CA 125 antibody was obtained from Fujirebio Diagnostics The antibody dialyzed against a solution of 50 mM sodium phosphate buffer, pH 7.0, and then filtered through a syringe filter (Gelman Acrodisc, 25 mm, 0.2p filter). The antibody concentration was determined spectrophotometrically. The antibody concentration was adjusted to 10 mg/ml with Ab buffer. The antibody was concentrated using an Amicon Stirred Cell and the final concentration was determined spectrophotometrically. For activation, the antibody was dispensed into a beaker and stirred in a temperature controlled water bath at 20° C. The activation reagent, succinimidyl 4-(N-maleimidomethyl)cyclohexane-1-carboxylate (SMCC), was dissolved in dimethyl formamide (DMF) to a concentration of 8.9 mg/mL The SMCC/DMF solution was added drop wise to the antibody while stirring such that the final concentration of SMCC was about 1 mg/ml. The antibody-SMCC solution was stirred gently for 60 minutes at 22° C. The activated antibody was filtered through one or more syringe filters into a clean amber glass vessel. The activated antibody solution was desalted that had previously been equilibrated with Desalt Buffer (100 mM sodium phosphate buffer, pH 6.0). The activated antibody was dialyzed against Antibody Buffer for 16-24 hours and transferred to an amber or foil wrapped glass bottle. The dialyzed antibody was then filtered through one or more syringe filters and the final concentration determined spectrophotometrically. The activated antibody was chromatographed on a Sephadex G₂₅ column using fast protein liquid chromatography (FPLC) (ÄTKA, GE Healthcare Life Sciences). The appropriate fractions containing activated antibody were pooled and the pooled antibody was filtered through one or more syringe filters. Antibody concentration was determined spectrophotometrically. Fractions containing activated were pooled and the pooled activated antibody was stored tightly capped in a temperature controlled water bath at 20° C. until the addition of activated HRP.

HRP activation. Horseradish peroxidase (HRP, Roche) was aliquoted into a glass bottle such that the ratio of HRP: antibody was 5:1 weight/weight plus 25% excess HRP. HRP was dissolved in HRP buffer (100 mM sodium phosphate buffer, pH 7.5) at a concentration of 20 mg/ml. The solution was dissolved by stirring at 20° C. in a temperature controlled water bath. N-succinimidyl-S-acetylthio-acetate (SATA) was aliquoted into a glass container and dissolved in DMF to a concentration of 50 mg/ml. The SATA/DMF solution was added drop wise to the HRP while stirring to a final SATA concentration of about 4.5 mg/mL. Hydroxylamine HCI Reagent (NH₂OH CI, Vitros) in a volume of 0.1 mL per mg of HRP was added to the HRP/SATA mixture drop wise with gentle stirring to a concentration such that final concentration of hydroxylamine (the active component of the hydroxylamine HCI reagent) was about 26.7 mg/ml and incubated for 15 minutes at 20° C. The activated HRP solution was chromatographed on a Sephadex G₂₅ column using fast protein liquid chromatography (FPLC) (ÄTKA, GE Healthcare Life Sciences). Fractions containing activated HRP or pooled, filtered through one or more syringe filters and the concentration was determined spectrophotometrically.

Antibody-HRP conjugation. Antibody-HRP conjugation was carried out in a Corning Low-FIow™ Reactor (from the Advanced-Flow series of reactors) that had been set up and equilibrated with Desalt Buffer at 25° C. the configuration was customized with the plate type and order: LFSIH, LFR*H, LFR*H, LFR*H, LFSHH, LFR*H, and LFSHH. For initial experiments, Pump A was set to 0.57 ml/min, Pump B was set to 1.43 ml/min, Pump C was set to 0.2 ml/min, and Pump D was set to 0.5 ml/min. The reactor was equilibrated with Quench and Stop solutions. The antibody solution was loaded into the reactor in Superloop A using Pump A. Horseradish peroxidase (HRP) was loaded into the reactor in Superloop B using Pump B. The conjugation reaction was run at a flow rate of 2.7 mL/min using the AKTA Unicorn System Control Software program. The reaction was monitored at 280 nm and 403 nm. The activated antibody and the activated HRP were allowed to flow through the conjugation reactor block for the times specified below. The conjugate solution then proceeded through a stop reactor block that had been equilibrated with beta mercaptoethanol stop solution using Pump C. From there, the conjugate proceeded through the Quench Reactor Block that had been equilibrated with Quench buffer using Pump D. The crude conjugate was collected and concentration was determined based on absorbance at 280 nm. The crude conjugate was concentrated using an Amicon stirred cell equipped with a YM30 membrane. The concentrated crude conjugate was filtered and then stored at 5° C. in a light protected class bottle. The concentrated crude conjugate was purified using a Sephacryl S300 HR column that had been equilibrated with Purification buffer using the AKTA FPLC purification system. Purified conjugate was either stabilized immediately or held for up to 24 hours at 2-8° C. prior to stabilization. The purified conjugate was stabilized by the addition of Proclin™ 300, potassium ferricyanide, and bovine serum albumin (BSA)

### Example 2

### Effect of Residence Time on Conjugate Yield

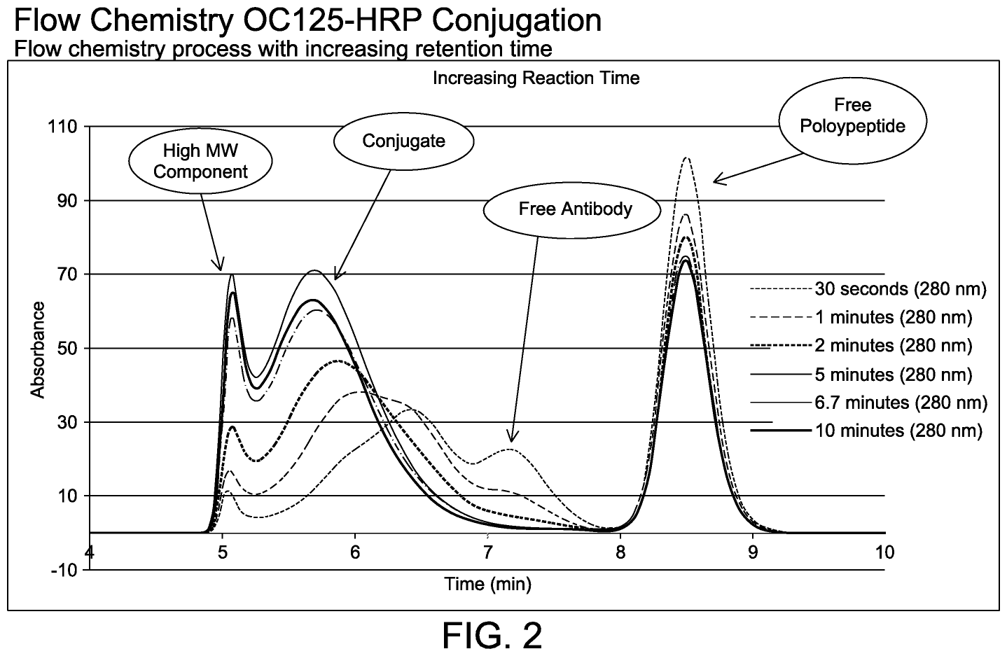

The antibody conjugation reactions using CA 125 antibody and horseradish peroxidase were carried out as described in Example 1 using residence times of 30 seconds, 1 minute, 2 minute, 5 minutes, 6.7 minutes, and 10 minutes. The reaction products were analyzed by size exclusion chromatography on an analytical HPLC column and absorbance at 280 nm was determined. Comparison of the reaction products for each condition is shown in FIG. 2. As shown in FIG. 2, longer residence times were correlated with increased production of high molecular weight component.

### Example 3

### Effect of HRP-Antibody Ratio on High Molecular Weight Product Yield

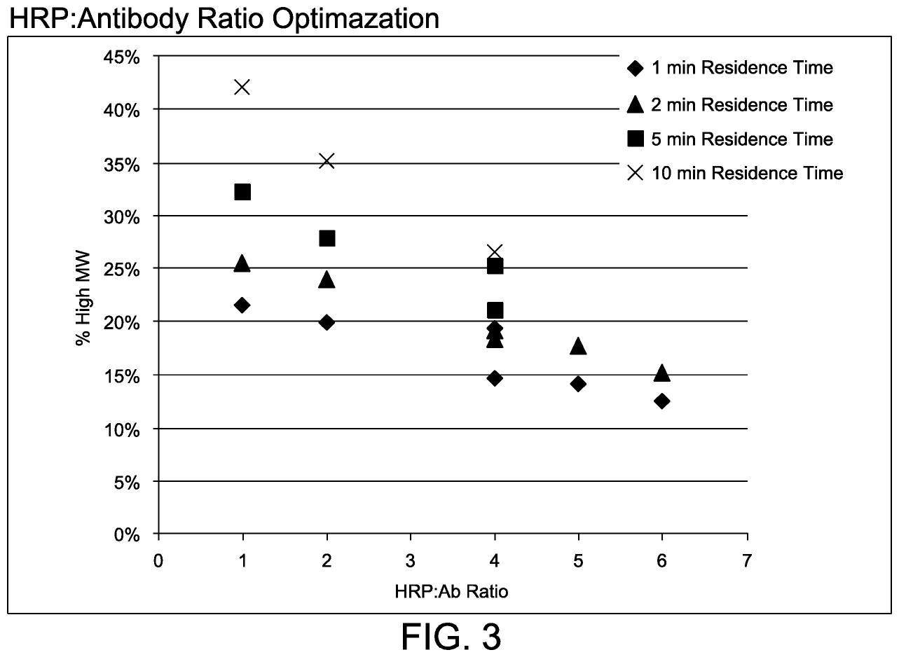

The antibody conjugation reactions using CA 125 antibody and horseradish peroxidase were carried out as described in Example 1 using HRP: antibody ratios of 1:1, 2:1, 4:1, 5:1, and 6:1. The conjugations were performed with residence times of 1 minute, 2 minute, 5 minutes, and 10 minutes. Comparison of the yield of high molecular weight products for each condition is shown in FIG. 3. As shown in FIG. 3, lower HRP: antibody ratios resulted in higher levels of high molecular weight products than did higher HRP: antibody ratios.

Thus, as shown in FIG. 2 and FIG. 3 the formation of high molecular weight species was dependent upon antibody: HRP ratio, antibody concentration, and residence time in the flow reactor.

### Example 4

### Conjugate Characterization

Percent HRP incorporation and Rz values (absorbance at 403 nm/absorbance at 280 nm) was determined for conjugates prepared using various ratios of HRP: antibody and various residence times. These values were compared with control conjugates prepared using standard batch methods. The percent HRP incorporation was estimated using the AUC values from HPLC traces. The results of this analysis are shown in the table below. As shown in the table the estimated HRP incorporation and RZ values were comparable to previous batch reactions. The percent HRP incorporation was determined based on the ratio of A₄₀₃ nm/A₂₈₀ nm.

TABLE 1
Estimated HRP incorporation and R_{z} values

| HRP:antibody | Residence time | | percent HRP |
|---|---|---|---|
| ratio | (minutes) | R_{z} | incorporation |
| 4:1 | 1 | 0.77 | 39% |
| 4:1 | 2 | 0.82 | 41% |
| 5:1 | 1 | 0.81 | 41% |
| 5:1 | 2 | 0.87 | 43% |
| 6:1 | 1 | 0.80 | 40% |
| 6:1 | 2 | 0.88 | 44% |
| Batch control | | 0.94 | 47% |

The conjugates were analyzed for batch to batch variability and stability. The conjugates prepared using the method above were comparable to those prepared using standard batch method techniques.

### Example 5

### Antibody Biotinylation

Mouse anti-human MUC1 antibody (DF3) antibody was obtained from Fujirebio Diagnostics. The antibody was dialyzed against a solution of 100 mM potassium phosphate buffer, pH 8.5. The antibody concentration was determined spectrophotometrically. The antibody concentration was adjusted to 10 mg/mL with 100 mM potassium phosphate buffer, pH 8.5. For the biotinylation, the antibody was dispensed into a beaker and stirred in a temperature-controlled water bath at 25° C. Biotin-e-aminocaproic acid N-hydroxy-succinimide-ester (Biotin-X-NHS) was dissolved in dimethyl sulfoxide (DMSO) at a concentration of 3.47 mg/ml. The Biotin-X-NHS/DMSO solution was added drop wise to the antibody solution while stirring such that the final concentration of Biotin-X-NHS was about 0.18 mg/ml. The antibody/Biotin-X-NHS solution was stirred gently for 90 minutes at 25° C. The reaction was quenched by drop wise addition of 1M Lysine solution, pH 8.5 to the antibody/Biotin-X-NHS solution while stirring such that the final concentration of Lysine was about 3 mg/mL. The antibody/Biotin-X-NHS/Lysine solution was stirred gently for 30 minutes at 25° C. The antibody/Biotin-X-NHS/Lysine solution was then dialyzed against a solution of 2 mM potassium phosphate buffer, pH 7.5 to purify the biotinylated antibody.

### Example 6

### Avidin-Biotinylated Antibody Conjugation

Avidin-Biotinylated Antibody conjugations were carried out in a Corning Low-Flow™ Reactor (from Advance-Flow series of reactors) that had been set up and equilibrated with 100 mM potassium phosphate buffer, pH 6.8 at 22° C. The configuration was customized with the plate type and order: LFSIH, LFR*H, LFR*H, LFR*H, and LFSHH. The biotinylated antibody solution was loaded into the reactor in Superloop A using Pump A. The avidin solution (Sigma Aldrich) was loaded into the reactor in Superloop B using Pump B. The unconjugated biotin quench solution was loaded into the reactor in Superloop C using Pump C. The conjugation reaction was run using the AKTA Unicorn System Control Software program. The reaction was monitored at 280 nm. The biotinylated-antibody and avidin were allowed to flow through the conjugation reactor block for the times specified below. The conjugate solution then proceeded through a quench reactor block that had been equilibrated with unconjugated biotin using Pump C.

### Example 7

### Effect of Residence Time on Conjugate Molecular Weight

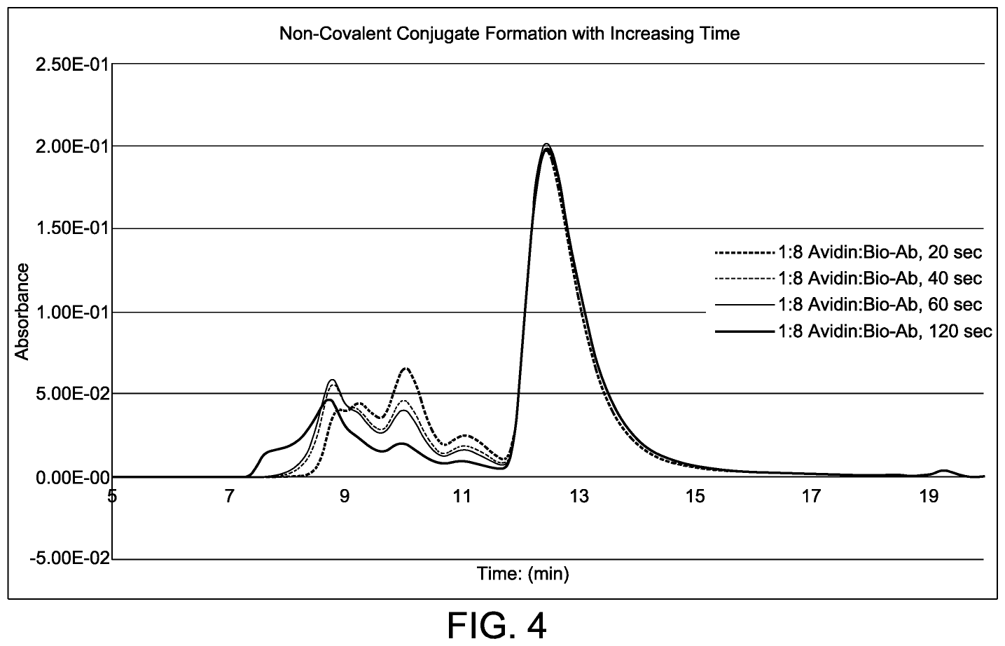

The antibody conjugation reactions using biotinylated antibody and avidin were carried out as described above using residence times of 20 seconds, 40 seconds, 60 seconds, and 120 seconds. The reaction products were analyzed by size exclusion chromatography on an analytical UPLC column and absorbance at 280 nm was determined. Comparison of the reaction products for each condition is shown in FIG. 2. As shown in FIG. 4, longer residence times were correlation with increased production of higher molecular weight conjugates.

### Example 8

### Effect of Avidin-Biotinylated Antibody on Conjugate Molecular Weight

The antibody conjugation reactions using biotinylated antibody and avidin were carried out as described in Examples 5-7 above using Avidin:Biotinylated Antibody ratios of 1:4, 1:6, 1:8, and 1:10. Comparison of the reaction products for each condition is shown in FIG. 6. As shown in FIG. 6, lower avidin:biotinylated antibody ratios resulted in higher levels of high molecular weight conjugates than did higher avidin:biotinylated antibody ratios.

1. A method of making an antibody-polypeptide conjugate, the method comprising:
(a) providing a first solution comprising an activated antibody;
(b) providing a second solution comprising an activated polypeptide;
(c) passing the first and second solutions through a continuous flow reactor, wherein the activated antibody contacts the activated polypeptide, thereby forming a covalent bond between the antibody and the polypeptide.

2. The method of claim 1, wherein the antibody is an IgG.

3. The method of claim 1, where in the antibody is an anti-CA₁₂₅ antibody.

4. The method of claim 1, wherein the polypeptide is a detection polypeptide

5. The method of claim 1, wherein the detection polypeptide is an enzyme, a biotin-binding polypeptide, or a fluorescent protein.

6. The method of claim 4, wherein the enzyme is selected from the group consisting of horseradish peroxidase, alkaline phosphatase, and urease.

7. The method of claim 1, wherein the antibody is activated via a succinimidyl ester, a hetero bifunctional reagent, a carbodiimide, or sodium periodate.

8. (canceled)

9. (canceled)

10. The method of claim 1, wherein the ratio of activated antibody: activated polypeptide is from 1:1 to about 1:4.

11. The method of claim 1, wherein the solutions are passed through the continuous flow reactor with a flow rate of between about 0.1 mL/min to about 10.0 mL/min.

12. (canceled)

13. The method of claim 11, wherein the flow rate provides a residence time of between about 30 seconds to about 15 minutes.

14. (canceled)

15. (canceled)

16. (canceled)

17. (canceled)

18. A method of making an antibody-polypeptide conjugate, the method comprising
(a) providing a first solution comprising a modified antibody;
(b) providing a second solution comprising a detection polypeptide, wherein the polypeptide specifically binds to the modified antibody;
(c) passing the solutions through a continuous flow reactor, wherein the modified antibody contacts the polypeptide, thereby forming a non-covalent bond between the antibody and the detection polypeptide.

19. The method of claim 18, wherein the modification is selected from the group consisting of antibody is biotinylation, carboxylation, phosphorylation, methylation, acetylation, nitrosylation, citrullination and deamination.

20. The method of claim 18, wherein the antibody is an IgG.

21. The method of claim 18, where in the antibody is an anti-CA₁₂₅ antibody.

22. The method of claim 18, wherein the detection polypeptide is an enzyme, a biotin-binding polypeptide, or a fluorescent protein.

23. The method of claim 22, wherein the enzyme is selected from the group consisting of horseradish peroxidase, alkaline phosphatase, and urease.

24. The method of claim 22, wherein the biotin binding polypeptide is streptavidin, avidin, neutravidin or an anti-biotin antibody.

25. The method of claim 18, wherein the ratio of activated antibody: detection polypeptide is from 1:1 to about 1:4.

26. The method of claim 18, wherein the solutions are passed through the continuous flow reactor with a flow rate of between about 0.1 mL/min to about 10.0 mL/min.

27. (canceled)

28. The method of claim 26, wherein the flow rate provides a residence time of between about 30 seconds to about 15 minutes.

29. (canceled)

30. (canceled)

31. (canceled)

32. (canceled)
